(19) United States
(12) Patent Application Publication
(10) Pub. No.: US 2006/0067907 A1
(43) Pub. Date: Mar. 30, 2006

(54) Cationic or cationizable polyurethane with elastic character

(76) Inventors: Nathalie Mougin, Paris (FR); Xavier Schultze, Pontault-Combault (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/230,670

(22) Filed: Sep. 21, 2005

Related U.S. Application Data
(60) Provisional application No. 60/620,671, filed on Oct. 22, 2004.

(30) Foreign Application Priority Data
Sep. 21, 2004 (FR) 04 52109

(51) Int. Cl. A61K 8/81

(52) U.S. Cl. 424/70.16; 424/70.17

(57) ABSTRACT

A cationic or cationizable polyurethane with elastic character formed by the reaction of: (a1) at least one cationic or cationizable unit resulting from at least one tertiary or quaternary amine having at least two reactive functions with labile H, (a2) at least one mixture of at least two different non-ionic units having at least two reactive functions with labile H, (b) at least one compound comprising at least two isocyanate functions, wherein the mixture of non-ionic units (a2) is comprised of a mixture of at least one first non-ionic polymer chosen from olefin homopolymers and copolymers, and at least one second non-ionic polymer different from an olefin copolymer and chosen from non-ionic homopolymers and copolymers, the first non-ionic polymer(s) and the second non-ionic polymer(s) bearing at their ends reactive functions with labile hydrogen and having a glass transition temperature (Tg), measured by differential scanning calorimetry, less than 10° C.

This application claims benefit of U.S. Provisional Application No. 60/620,671, filed Oct. 22, 2004, the contents of which are incorporated herein by reference. This application also claims benefit of priority under 35 U.S.C. § 119 to French Patent Application No. 04 52109, filed Sep. 21, 2004, the contents of which are also incorporated by reference.

The present disclosure relates to novel cationic or cationizable polyurethanes with elastic character as well as their use in cosmetic compositions.

The formation of deposits and films with elastic properties has always been the subject of much research in the cosmetic arts. Indeed, most of the areas of the human body able to receive cosmetic deposits, such as skin, lips, hair, eyelashes and nails, are subjected to substantial mechanical deformations and stresses. Ideally, cosmetic films and deposits should be able to resist these stresses and follow these deformations without breaking.

The use of polyurethanes in cosmetics has been known for a long time and is described, for example, in patent documents WO 94/13724 and EP 0 619 111.

The polyurethanes-disclosed in these documents have, however, glass transition temperatures (Tg) higher than room temperature (20° C.), i.e. at room temperature, they may be in a glassy state and may form brittle films unacceptable for cosmetic applications.

Admittedly, there are physiologically acceptable polymers having low glass transition temperatures, such as, for example, acrylic polymers, but these polymers generally form very tacky deposits, which present a disadvantage in most cosmetic applications.

Furthermore, physiologically acceptable polyurethanes which form non-tacky, non-brittle films and are capable of plastic and elastic deformations are known from International Application WO 02/32978. These interesting viscoelastic properties are believed to be due to the presence, in the polymer, of long macromolecular units having a relatively low glass transition temperature and which thereby, at room temperature, are not in glassy state.

Thus, in view of known polyurethanes discussed above, the present disclosure provides physiologically acceptable polyurethanes having improved film forming and viscoelastic properties.

Disclosed herein, therefore, is a cationic or cationizable polyurethane with elastic character, comprising at least a cationic or cationizable unit, and at least two non-ionic units.

For example, the cationic or cationizable polyurethane with elastic character is formed by the reaction of:

(a1) at least one cationic or cationizable unit resulting from at least one tertiary or quaternary amine having at least two reactive functions with labile H,

(a2) at least one mixture of at least two different non-ionic units having at least two reactive functions with labile H,

(b) at least one compound comprising at least two isocyanate functions,

wherein the mixture of non-ionic units (a2) comprises at least one mixture of at least one first non-ionic polymer chosen from olefin homopolymers and copolymers, and at least one second non-ionic polymer different from an olefin copolymer and chosen from non-ionic homopolymers and copolymers, the first non-ionic polymer(s) and the second non-ionic polymer(s) bearing at their ends reactive functions with labile hydrogen and having a glass transition temperature (Tg), measured by differential scanning calorimetry, less than 10° C.

As used herein, “cationic or cationizable unit” means any unit which, either by its own chemical nature, or depending on the medium and/or the pH in which it is, will be in cationic form.

Also disclosed herein is the use of cationic or cationizable polyurethane with elastic character as defined above in cosmetic compositions in view of improving the viscoelastic properties of cosmetic deposits and films obtained from these compositions.

Further disclosed herein is the use of this polyurethane in lacquers and styling compositions, nail polishes, skin, lip and skin appendage make-up compositions, and to form protecting films for nails.

The present disclosure also provides for the use of this polyurethane for styling hair and, for example, for the formulation of lacquers, styling mousses or gels.

The present disclosure further provides for cosmetic compositions comprising cationic or cationizable polyurethane with elastic character as defined above.

The polyurethane with elastic character according to the present disclosure, thanks to its cationizable character, has, in at least one embodiment, the advantage of having an excellent affinity to keratin substrates such as hair, nails and stratum corneum epidermidis to which the keratin confers a negative charge.

The use of cationic or cationizable polyurethane with elastic character according to the present disclosure in lacquers and styling compositions may, for example, allow for the improvement of hairstyle flexibility, i.e., to achieve a more natural and lasting hair hold than that which may be obtained with fixing polymers of the prior art.

The polyurethane of the present disclosure may be used to coat the nails with a glossy protecting film resistant to mechanical stresses. Its incorporation in a nail polish may enhance the impact-resistance thereof and delay chipping.

The cationic or cationizable polyurethane according to the present disclosure can also be used to improve the hold of skin, lip, and skin appendage make-up compositions. Indeed, make-up products containing the polyurethane disclosed herein may adhere well to the skin and skin appendages and the obtained deposits may follow the deformations of keratin substrates and thus not pull the skin.

In all of these applications, non-tacking products are, for example, obtained.

Moreover, the presently disclosed compositions comprising polyurethane may have improved moisture resistance through the use of olefin copolymers which have hydrophobic character.

As used herein, “elastic material” means a macromolecular material which returns rapidly to its initial shape and dimensions after releasing a weak stress that has produced a substantial deformation.

As shown above, the cationic or cationizable polyurethane with elastic character disclosed herein comprises at least one cationic or cationizable unit, and at least two non-ionic units.

As noted above, disclosed herein is a cationic or cationizable polyurethane with elastic character formed by the reaction of:

(a1) at least one cationic or cationizable unit resulting from at least one tertiary or quaternary amine having at least two reactive functions with labile H,

(a2) at least one mixture of at least two different non-ionic units having at least two reactive functions with labile H,

(b) at least one compound comprising at least two isocyanate functions,

wherein the mixture of non-ionic units (a2) comprises a mixture of at least one first non-ionic polymer chosen from olefin homopolymers and copolymers, and at least one second non-ionic polymer different from a olefin homopolymer or copolymer and chosen from non-ionic homopolymers and copolymers, the first non-ionic polymer(s) and the second non-ionic polymer(s) bearing at their ends reactive functions with labile hydrogen and having a glass transition temperature (Tg), measured by differential scanning calorimetry, less than 10° C.

By “reactive functions with labile hydrogen,” as used herein is meant functions capable, after a hydrogen atom has left, of forming covalent bonds with the isocyanate functions of compounds comprising at least two isocyanate functions. Examples of such functions include hydroxyl, primary or secondary amine groups, or thiol groups.

The polycondensation of compounds bearing these labile hydrogen reactive functions with compounds comprising at least two isocyanate functions yields, according to the nature of the reactive functions bearing the labile hydrogen (—OH, —NH₂, —NHR or —SH), polyurethanes, polyureas or polythiourethanes, respectively. Thus, the polymers used herein may be a copoly urethane/urea and/or a thiourethane.

All of these polymers are grouped together herein, as a matter of simplification, under the term polyurethanes.

According to at least one embodiment, the at least one first non-ionic olefin copolymer and the at least one second polymer different from a non-ionic olefin copolymer forming the non-ionic units (a2) have a glass transition temperature, measured by differential scanning calorimetry, less than 0° C., such as less than −10° C.

For example, the olefin homopolymers and copolymers may be homopolymers and copolymers having units chosen from ethylene, propylene, 1-butylene (or 1-butene), 2-butylene (or 2-butene), isobutylene, 1,2-butadiene, 1,4-butadiene and isoprene units.

When the olefin polymers result from the polymerization of butadiene, they may be copolymers (1,2-butadiene, 1,4-butadiene).

Olefin homopolymers or copolymers resulting from the polymerization of butadiene can undergo, subsequent to the polymerization, a hydrogenation of the residual double bonds. This hydrogenation may be total or partial.

In the case of partial hydrogenation, the content of residual double bonds remains low. These polymers are, however, termed ethylene and butylene copolymers, as a matter of simplification.

As a first non-ionic polymer, the olefin copolymer in at least one embodiment is ethylene and butylene copolymer. These include, for instance, KRATON L, and, for example, KRATON L2203 sold by the KRATON company, and G13000 sold by the NISSO CHEMICAL company.

As a polyolefin copolymer, random copolymers (1,2-butadiene/1,4-butadiene) can be further mentioned, for example.

In addition to the first non-ionic polymer(s) chosen from olefin homopolymers and copolymers, (a2) units are also chosen from non-ionic homopolymers and copolymers, but those which are different from an olefin polymer, i.e., non-olefin non-ionic homopolymers and copolymers.

The homopolymers and/or copolymers different from an olefin polymer which form the non-ionic units (a2) are generally chosen from, for example, poly(meth)acrylates, polyamides, hydrocarbons, fluorinated or perfluorinated, polyethers, polyesters, polyalkylsiloxanes and hydrogenated, fluorinated or perfluorinated polycarbonates.

In at least one embodiment, polyethers can be chosen from poly(C₂-C₄ alkylene oxide) with number-average molecular weight ranging from 400 to 50,000, for instance poly(tetramethylene oxide). The poly(tetramethylene oxide) include, for example, polymers sold under the name TERATHANE by the Dupont de Nemours Company. In at least one embodiment, poly(tetramethylene oxide) having a number-average molecular weight of 1400 or 2900 is used.

According to at least one embodiment, the unit (a2) mixture is a mixture of poly(tetramethylene oxide) and at least one copolymer having ethylene, 1,2-butadiene and/or 1,4-butadiene units.

Generally, the first non-ionic olefin polymer(s) and the second non-olefin polymer(s) forming the non-ionic units (a2) each have a number-average molecular weight ranging from 400 to 50,000, such as from 400 to 30,000, or from 400 to 25,000.

In addition to non-ionic unit(s) (a2), the cationic or cationizable polyurethane according to the present disclosure is comprised of at least one cationic or cationizable unit (a1) resulting from at least one tertiary or quaternary amine having at least two reactive functions with labile hydrogen.

When the tertiary or quaternary amine(s) forming the units (a1) bear more than two functions with labile hydrogen, the resulting polyurethane has a branched structure.

According to at least one embodiment, the tertiary or quaternary amine(s) forming the cationic or cationizable units (a1) have only two reactive functions with labile hydrogen, and therefore, the polyurethanes obtained by polycondensation have an essentially linear structure.

It is also possible, for example, to use a bifunctional amine mixture containing a low proportion of amines bearing more than two reactive functions with labile hydrogen.

The tertiary or quaternary amine(s) forming the cationic or cationizable units (a1) are, in at least one embodiment, chosen from the compounds corresponding to one of the following formulas: 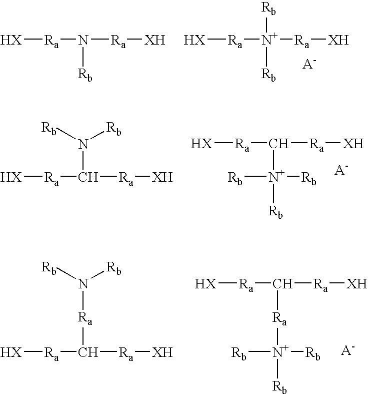
wherein

each Rₐ is independently chosen from linear and branched C₁-C₆ alkylene groups, c₃-C₆ cycloalkylene groups, arylene groups, and mixtures thereof; all of which may be substituted by at least one halogen atom and may include at least one heteroatom chosen from O, N, P and S,

each R_{b} is independently chosen from C₁-C₆ alkyl groups, C₃-C₆ cycloalkyl groups, aryl groups, and mixtures thereof; all of which may be substituted by at least one halogen atom and may include at least one heteroatom chosen from O, N, P and S,

each X is independently an entity chosen from an oxygen atom, a sulphur atom, an NH group, and an NR_{c} group, where R_{c} is a C₁-C₆ alkyl group, and

A⁻ is a physiologically acceptable counter-ion.

In at least one embodiment, tertiary amines for obtaining the cationic or cationizable polyurethane with elastic character of the present disclosure include N-methyl diethanolamine and N-tert-butyl diethanolamine.

The tertiary and quaternary amine(s) forming the cationic or cationizable units (a1) of the polyurethane disclosed herein may also be polymers with tertiary and/or quaternary amine function(s), bearing at their ends reactive functions with labile hydrogen chosen from —OH, —NH₂, —NHR_{c} or —SH, where R_{c} represents a C₁-C₆ alkyl group. The average weight molecular weight of these polymers with tertiary and/or quaternary amine functions ranges from 400 to 10,000.

Examples of such appropriate polymers with amine function(s) include polyesters resulting from the polycondensation of N-methyl diethanolamine and adipic acid.

When the amines forming cationic or cationizable units (a1) are compounds with tertiary amine function(s), a part or all of these amine functions must be neutralized by a suitable neutralizing agent chosen from physiologically acceptable organic or mineral acids. Examples of acids that may be used include hydrochloric acid, acetic acid and tartaric acid.

As previously explained, the polyurethane according to the present disclosure is formed by the reaction of compounds (a1) and (a2) with at least a compound (b) comprising at least two isocyanate functions.

In at least one embodiment, the compound(s) comprising at least two isocyanate functions are chosen from diisocyanates or mixtures of a diisocyanate and an isocyanate comprising more than two isocyanate functions, said isocyanate representing from 0.1 to 10% by weight of the weight of said mixture.

The compound(s) comprising at least two isocyanate functions may be chosen from aliphatic, cyclic diisocyanates, conjugated or not, aromatic or not.

Thus, the diisocyanate(s) of at least one embodiment are chosen, for example, from methylene diphenyl diisocyanate, methylene cyclohexane diisocyanate, isophorone diisocyanate, toluene diisocyanate, naphthalene diisocyanate, 1,4-butane diisocyanate and 1,6-hexane diisocyanate.

The cationic or cationizable polyurethane with elastic character of the present disclosure may contain, in addition to (a1) and (a2) units, at least one non-ionic monomer unit (a3) comprising at least two functions with labile hydrogen capable of reacting with said compound(s) (b) comprising two isocyanate functions.

In at least one embodiment, the non-ionic monomer compounds forming the non-ionic units (a3) are chosen from C₁-C₁₂ diols, such as neopentyl glycol, hexa(ethylene glycol), 1,2-ethanediol, 1,2-propanediol and 1,3-propanediol, and C₁-C₆ aminoalcohols, such as aminoethanol.

The viscosity of the cationic or cationizable polyurethane according to the present disclosure, measured at 10% in tetrahydrofuran, at 25° C., with a Brookfield viscosimeter, needle module, is generally in the range of 1 to 1000 cps, such as 1 to 100 cps, for instance 2 to 80 cps. The polyurethane is characterized in non neutralized form.

The physical parameter characterizing the viscoelastic properties of the previously described cationic or cationizable polyurethane is its tensile recovery. This recovery is determined by a tensile creep test which includes rapidly stretching a specimen to a predetermined elongation rate, and then releasing the stress, and measuring the specimen length.

The creep test used for characterizing the cationic or cationizable polyurethane with elastic character according to the invention is as follows:

as a specimen, a film of the polyurethane having a thickness of 500±50 mm, cut into strips of 80 mm×15 mm is used. This copolymer film is obtained by drying, at 22±2° C. and at a relative humidity of 50±5%, a solution or dispersion at 3% by weight of said polyurethane in water and/or ethanol.

Each strip is fixed between two jaws sited 50±1 mm apart, and is stretched at a rate of 20 mm/minute (under the above temperature and relative humidity conditions) to an elongation of 50% (εₘₐₓ), i.e. up to 1.5 times its initial length. The stress is then released by imposing a back speed equal to the tensile speed, that is 20 mm/minute, and the specimen elongation is measured (as a % in relation to the initial length) immediately after return to zero load (εᵢ).

The instantaneous recovery (Rᵢ) is calculated by means of the following formula:
Rᵢ(%)=((εₘₐₓ−εᵢ)/εₘₐₓ)×100

The cationic or cationizable polyurethane with elastic character of the present disclosure has, in at least one embodiment, an instantaneous recovery (Rᵢ), measured under the conditions set forth above, in the range of 5% to 95%, such as in the range of 20% to 90%, for example, 35 to 85%.

The glass transition temperature (Tg) of the non-ionic polymers forming the (a2) units and the cationic or cationizable polyurethane of the present disclosure is measured by differential scanning calorimetry (DSC) according to the ASTM standard D3418-97.

The cationic or cationizable polyurethane with elastic character disclosed herein has, in at least one embodiment, at least two glass transition temperatures, at least one of which is less than 10° C., such as less than 0° C. and, for example, less than −10° C., and at least another one is greater than or equal to room temperature (20° C.).

The instantaneous recovery, and therefore, the viscoelastic properties of the polyurethane disclosed herein, depends on the fractions of the different monomer units (a1), (a2), (a3) and on the amount of compound (b) comprising at least two isocyanate functions.

The (a1) unit fraction, in at least one embodiment, is sufficient to impart to polymers their positive charge responsible for their good affinity to keratin substrates. In at least one embodiment, the (a2) unit(s) are present in a sufficient fraction by weight so that the polyurethanes present at least a glass transition temperature less than 10° C. and form no brittle films.

Generally, the cationic or cationizable units (a1) represent from 0.1 to 90%, such as from 1 to 30%, or from 1 to 20% by weight; the non-ionic units (a2) from 10 to 99.9%, such as from 20 to 99%, or from 30 to 85% by weight; and (a3) units from 0 to 50% by weight, such as from 0 to 40%, or from 0 to 30% by weight of the cationic or cationizable polyurethane.

The compound(s) (b) comprising at least two isocyanate functions are present in an essentially stoichiometric amount as compared to the sum of the tertiary or quaternary amine(s) forming the (a1) units, the first non-ionic polymer(s) and the second non-ionic polymer(s) forming (a2) units, and the non-ionic monomer compound(s) forming the (a3) units.

Indeed, obtaining polyurethanes having significant molecular weights supposes a number of isocyanate functions virtually identical to the number of functions with labile hydrogen. One of skill in the art will know how to select a possible molar excess of the either type of function in order to adjust the molecular weight at the desired value.

As shown above, the cationic or cationizable polyurethane with elastic character according to the present disclosure may be included in many cosmetic compositions where it can improve the cosmetic properties.

The amount of polyurethane present in the different compositions depends of course on the composition type and the desired properties and may vary within a very wide range, generally from 0.1 to 90% by weight, such as from 1 to 50% by weight, based on the final cosmetic composition.

When the cationic or cationizable polyurethane with elastic character is included in hair lacquers, it is present generally in the range of 0.5 to 15% by weight. In nail polishes, it generally represents from 0.5 to 40% by weight of the composition. In make-up compositions for skin, lips and skin appendages, it generally represents from 0.5 to 20% by weight of the composition. In hair styling product compositions, it generally represents from 0.5 to 10% by weight of the composition. In shampoo compositions, it generally represents from 0.1 to 5% by weight of the composition.

The composition disclosed herein may further comprise at least one cosmetically acceptable adjuvant conventionally used in cosmetic compositions for the application on keratin fibers. These include, for instance, gelling and/or thickening agents such as polymeric thickenings, associative or not, anionic, non-ionic, cationic or amphoteric surfactants, propenetrants, emulsifying agents, perfumes, preservatives, fillers, sunscreens, dyestuffs, proteins, vitamins, provitamins, anionic, non-ionic, cationic or amphoteric non fixing polymers, moisturizing agents, emollients, softener agents, mineral, vegetal or synthetic oils, hydrophilic or lipophilic actives such as ceramides and pseudoceramides, antifoam agents, antiperspirants, anti-radical agents, fixing or non fixing polymers; bactericidal agents and antidandruff agents.

The composition according to the present disclosure may be in the form of lotion, thickened or not, cream, thickened or not, gel, mousse or any other suitable form. It can optionally be packaged in a pump package or in an aerosol container.

Also disclosed herein is the use of the above-described cationic or cationizable polyurethane with elastic character to form a protecting film on nails and its use in rinsed application on hair.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

The following examples illustrate certain embodiments of the present disclosure in a non-limiting manner:

### EXAMPLE 1

### Synthesis of Cationic or Cationizable Polyurethane with Elastic Character

The synthesis of cationic or cationizable polyurethane with elastic character as disclosed herein was achieved by the reaction of the following compounds (by weight):

a1) 8.6% of N-methyl diethanolamine (noted NMDEA),

a2) 8.4% of a poly(tetramethylene oxide) (noted PTMO) of weight average molecular weight equal to 1400, and 61.6% by weight of ethylene-butylene copolymer sold under the reference KRATON L2203 (of weight average molecular weight of 2000) by the KRATON company,

b) 21.4% by weight of isophorone diisocyanate (noted IPDI).

1. Procedure:

To a 500 ml reactor equipped with a condenser and a mechanical stirring, PTMO (8.4 g), KRATON L2203 (61.6 g) and NMDEA (8.6 g) in 100 g of THF (the solid being equal to 44% by weight) were added.

The reaction medium was homogenized with stirring at 100 rpm with THF reflux for 45 minutes. The reaction medium temperature was 80° C.

The IPDI (21.4 g) and 20 g of THF were introduced in an addition funnel which was fixed on the reactor. This mixture was added to the reaction medium within 45 minutes.

After the mixture was added, the medium was allowed to reflux for 90 minutes.

0.1 g of dibutyltin dilaurate was then added, resulting in a short time reflux increase (5 min).

The reaction medium was allowed to reflux for 210 minutes after which the characteristic peak of NCO moieties had disappeared in infrared (2250 cm⁻¹). A progressive increase of the viscosity was observed.

10 g of ethanol were added and allowed further 60 minutes to reflux in order to eliminate any traces of IPDI which would not be detected by infrared.

2. Aqueous Dispersing:

At room temperature, the reaction medium was diluted with THF until a solid of 30% by weight was obtained.

72 ml of 1 N hydrochloric acid were added with mechanical stirring (200 rpm), allowing 100% neutralization of tertiary amine functions. A high increase of the viscosity was observed.

400 g of water were then added dropwise by means of an addition funnel, leading to a whitening of the medium and a decrease of the viscosity.

The thus obtained mixture was then evaporated on a rotovapor at a bath temperature of 50° C., by progressively increasing the void so as to eliminate the solvent.

A polyurethane dispersed in water was obtained. The particle size was 160 nm. The solid was 29.6% by weight. The pH was 4.65.

The particle sizes were measured by light scattering using a Coulter N4 SD granulometer.

The solid was measured by putting samples in a ventilated oven at 70° C. overnight.

The viscosity of the non neutralized form at 10% in THF at 25° C. was 20 cps.

The elasticity was tested as follows: a specimen was made from a film achieved by drying the aqueous dispersion. A 50% instantaneous elastic recovery was obtained after 100% deformation.

### EXAMPLE 2

### Synthesis of Cationic or Cationizable Polyurethane with Elastic Character

The synthesis of cationic or cationizable polyurethane with elastic character disclosed herein achieved by the reaction of the following compounds (by weight):

a1) 8.6% of N-methyl diethanolamine (noted NMDEA),

a2) 8.5% of a poly(tetramethylene oxide) (noted PTMO) of weight average molecular weight equal to 1400, and 61.6% by weight of ethylene-butylene copolymer sold under the reference GI 3000 (of weight average molecular weight of 3000) by the NISSO CHEMICAL company,

b) 21.3% by weight of isophorone diisocyanate (noted IPDI).

The procedure was the same as that described in example 1, except the solvent used for the synthesis and the dispersion, which was methyl ethyl ketone. The neutralization of the tertiary amine functions was performed only at 75% and not at 100% as in example 1.

A cationic or cationizable polyurethane with elastic character dispersed in water, having the following characteristics was obtained:

Particle size: 700 nm

Solid: 10%

pH=5

Viscosity: between 5 and 20 cps

Instantaneous elastic recovery after 100% deformation: 70%.

### EXAMPLE 3

### Styling Gel Comprising a Polyurethane According to the Invention

A styling gel composition comprising the polyurethane according to example 1 was formulated.

The composition had the following formulation (by weight based on the total weight of the composition):

| | |
|---|---|
| Polyurethane as disclosed herein | 4% |
| Jaguar HP 105 | |
| (hydroxypropyl guar, sold by the RHODIA company) | 4% |
| Water | 4% |

Applied on hair, this gel provided the hair with easy and flexible hair styling.

### EXAMPLE 4

### Styling Mousse Comprising a Polyurethane According to the Invention

A styling composition comprising the polyurethane according to example 1 was formulated.

The composition had the following formulation (by weight based on the total weight of the composition):

| | |
|---|---|
| Polyurethane as disclosed herein | 2% |
| Oxyethylene sorbitan monolaurate (20 OE) | 0.2% |
| Water | 92.8% |
| Isobutane/propane/butane (50/25/25) | 5% |

Applied on hair by means of aerosol, this mousse provided thair with an easy and flexible styling.

What is claimed is:

1. A cationic or cationizable polyurethane with elastic character formed by the reaction of:
(a1) at least one cationic or cationizable unit resulting from at least one tertiary or quaternary amine having at least two reactive functions with labile H,
(a2) at least one mixture of at least two different non-ionic units having at least two reactive functions with labile H,
(b) at least one compound comprising at least two isocyanate functions,
wherein the at least one mixture of non-ionic units (a2) comprises a mixture of
at least one first non-ionic polymer chosen from olefin homopolymers and copolymers, and
at least one second non-ionic polymer different from an olefin copolymer and chosen from non-ionic homopolymers and copolymers, and
further wherein said at least one first non-ionic polymer and said at least one second non-ionic polymer bear at their ends reactive functions with labile hydrogen and having a glass transition temperature (Tg), measured by differential scanning calorimetry, less than 10° C.

2. The cationic or cationizable polyurethane with elastic character according to claim 1, wherein said at least one first non-ionic olefin polymer and said at least one second non-ionic polymer forming the non-ionic units (a2) have a glass transition temperature, measured by differential scanning calorimetry, less than 0° C.

3. The cationic or cationizable polyurethane with elastic character according to claim 2, wherein said glass transition temperature is less than −10° C.

4. The cationic or cationizable polyurethane with elastic character according to claim 1, wherein the olefin homopolymers and copolymers are homopolymers and copolymers having units chosen from ethylene, propylene, 1-butylene, 2-butylene, isobutylene, 1,2-butadiene, 1,4-butadiene and isoprene units.

5. The cationic or cationizable polyurethane with elastic character according to claim 1, wherein the at least one first non-ionic polymer is an ethylene and butylene copolymer.

6. The cationic or cationizable polyurethane with elastic character according to claim 1, wherein the non olefin homopolymers and/or copolymers forming the non-ionic units (a2) are chosen from poly(meth)acrylates, polyamides, hydrocarbon, fluorinated or perfluorinated polyethers, polyesters, polyalkylsiloxanes and hydrogenated, fluorinated or perfluorinated polycarbonates.

7. The cationic or cationizable polyurethane with elastic character according to claim 6, wherein the polyethers are chosen from poly(C₂-C₄ alkylene oxide) of number-average molecular weight ranging from 400 to 50,000.

8. The cationic or cationizable polyurethane with elastic character according to claim 7, wherein the polyethers are chosen from poly(tetramethylene oxide) of number-average molecular weight ranging from 400 to 50,000.

9. The cationic or cationizable polyurethane with elastic character according to claim 1, wherein the non-ionic units (a2) result from the reaction of the mixture of poly(tetramethylene oxide) and copolymer having ethylene, 1,2-butadiene and/or 1,4-butadiene units.

10. The cationic or cationizable polyurethane with elastic character according to claim 1, wherein the at least one first olefin polymer and the at least one second non olefin polymer forming the non-ionic units (a2) each have a number-average molecular weight ranging from 400 to 50,000.

11. The cationic or cationizable polyurethane with elastic character according to claim 10, wherein the at least one first olefin polymer and the at least one second non olefin polymer forming the non-ionic units (a2) have a number-average molecular weight ranging from 400 to 30,000.

12. The cationic or cationizable polyurethane with elastic character according to claim 11, wherein the at least one first olefin polymer and the at least one second non olefin polymer forming the non-ionic units (a2) have a number-average molecular weight ranging from 400 to 25,000.

13. The cationic or cationizable polyurethane with elastic character according to claim 1, wherein the non-ionic units (a2) represent from 10 to 99.9% by weight of the cationic or cationizable polyurethane.

14. The cationic or cationizable polyurethane with elastic character according to claim 13, wherein the non-ionic units (a2) represent from 20 to 99% by weight of the cationic or cationizable polyurethane.

15. The cationic or cationizable polyurethane with elastic character according to claim 14, wherein the non-ionic units (a2) represent from 30 to 85%, by weight of the cationic or cationizable polyurethane.

16. The cationic or cationizable polyurethane with elastic character according to claim 1, wherein the at least one cationic or cationizable unit (a1) results from an amine chosen from amines having the following formulas: 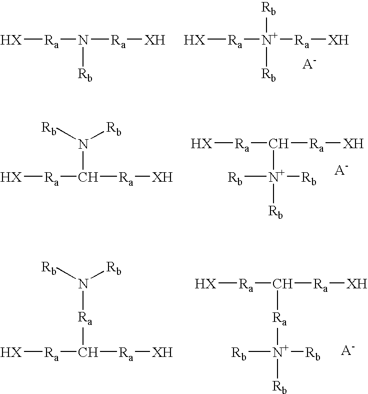
wherein
each Rₐ is independently chosen from a linear or branched C₁-C₆ alkylene group, a C₃-C₆ cycloalkylene group and an arylene group, and mixtures thereof; all of which may be substituted by at least one halogen atom and which may include at least one heteroatom chosen from O, N, P and S,
each R_{b} is independently chosen from a C₁-C₆ alkyl group, a C₃-C₆ cycloalkyl group and an aryl group, and mixtures thereof; all of which may be substituted by at least one halogen atom and which may include at least one heteroatom chosen from O, N, P and S,
each X is independently an entity chosen from an oxygen atom, a sulphur atom, a NH group, and a NR_{c} group, where R_{c} is a C₁-C₆ alkyl group, and
A⁻ is a physiologically acceptable counter-ion.

17. The cationic or cationizable polyurethane with elastic character according to claim 16, wherein the at least one cationic or cationizable unit (a1) results from the reaction of N-methyl diethanolamine or tert-butyl diethanolamine.

18. The cationic or cationizable polyurethane with elastic character according to claim 16, wherein the (a1) units result from the reaction of at least one polymer having at least one function chosen from tertiary and quaternary amine functions, bearing at their ends reactive functions with labile hydrogen chosen from —OH, —NH₂, —NHR_{c} or —SH, and having a weight-average molecular weight ranging from 400 to 10,000, wherein R_{c} is a C₁-C₆ alkyl group.

19. The cationic or cationizable polyurethane with elastic character according to claim 1, wherein the at least one cationic or cationizable unit (a1) represents from 0.1 to 90% by weight of the cationic or cationizable polyurethane.

20. The cationic or cationizable polyurethane with elastic character according to claim 19, wherein the at least one cationic or cationizable unit (a1) represents from 1 to 30% by weight of the cationic or cationizable polyurethane.

21. The cationic or cationizable polyurethane with elastic character according to claim 20, wherein the at least one cationic or cationizable unit (a1) represents from 1 to 20% by weight of the cationic or cationizable polyurethane.

22. The cationic or cationizable polyurethane with elastic character according to claim 1, wherein the at least one compound (b) comprising at least two isocyanate functions is chosen from diisocyanates or mixtures of a diisocyanate and an isocyanate comprising more than two isocyanate functions, said isocyanate representing from 0.1 to 10% by weight of the weight of said mixture.

23. The cationic or cationizable polyurethane with elastic character according to claim 22, wherein the diisocyanates are chosen from methylene diphenyl diisocyanate, methylene cyclohexane diisocyanate, isophorone diisocyanate, toluene diisocyanate, naphthalene diisocyanate, 1,4-butane diisocyanate and 1,6-hexane diisocyanate.

24. The cationic or cationizable polyurethane with elastic character according to claim 1, wherein it optionally further comprises at least one non-ionic unit (a3) containing at least two functions with labile hydrogen capable of reacting with said at least one compound (b) comprising two isocyanate functions.

25. The cationic or cationizable polyurethane with elastic character according to claim 24, wherein the at least one non-ionic unit (a3) is formed from at least one monomer compound chosen from C₁-C₁₂ diols, preferably neopentyl glycol, hexa(ethylene glycol), 1,2-ethanediol, 1,2-propanediol and 1,3-propanediol, and C₁-C₆ aminoalcohols, preferably aminoethanol.

26. The cationic or cationizable polyurethane with elastic character according to claim 25, wherein the C₁-C₁₂ diols are chosen from neopentyl glycol, hexa(ethylene glycol), 1,2-ethanediol, 1,2-propanediol and 1,3-propanediol.

27. The cationic or cationizable polyurethane with elastic character according to claim 25, wherein the C₁-C₆ aminoalcohols are chosen from aminoethanol.

28. The cationic or cationizable polyurethane with elastic character according to claim 24, wherein the at least one non-ionic unit (a3) represents from 0 to 50% by weight of the cationic or cationizable polyurethane.

29. The cationic or cationizable polyurethane with elastic character according to claim 28, wherein the at least one non-ionic unit (a3) represents from 0 to 40% by weight of the cationic or cationizable polyurethane.

30. The cationic or cationizable polyurethane with elastic character according to claim 29, wherein the at least one non-ionic unit (a3) represents from 0 to 30% by weight of the cationic or cationizable polyurethane.

31. The cationic or cationizable polyurethane with elastic character according to claim 1, wherein it has at least two different glass transition temperatures (Tg), at least one of these Tg being less than 10° C. and at least another being greater than or equal to 20° C.

32. The cationic or cationizable polyurethane with elastic character according to claim 1, wherein it has an instantaneous recovery value ranging from 5 to 95%.

33. The cationic or cationizable polyurethane with elastic character according to claim 32, wherein it has an instantaneous recovery value ranging from 20 to 90%.

34. The cationic or cationizable polyurethane with elastic character according to claim 33, wherein it has an instantaneous recovery value ranging from 35 to 85%.

35. The cationic or cationizable polyurethane with elastic character according to claim 1, wherein it has a viscosity ranging from 1 to 100 cps.

36. A cosmetic composition comprising, in a cosmetically acceptable medium, a cationic or cationizable polyurethane with elastic character formed by the reaction of:
(a1) at least one cationic or cationizable unit resulting from at least one tertiary or quaternary amine having at least two reactive functions with labile H,
(a2) at least one mixture of at least two different non-ionic units having at least two reactive functions with labile H,
(b) at least one compound comprising at least two isocyanate functions, wherein the at least one mixture of non-ionic units (a2) comprises a mixture of
at least one first non-ionic polymer chosen from olefin homopolymers and copolymers, and
at least one second non-ionic polymer different from an olefin copolymer and chosen from non-ionic homopolymers and copolymers, and
further wherein said at least one first non-ionic polymer and said at least one second non-ionic polymer bear at their ends reactive functions with labile hydrogen and having a glass transition temperature (Tg), measured by differential scanning calorimetry, less than 10° C.

37. The cosmetic composition according to claim 36, wherein it is a hair lacquer and comprises from 0.5 to 15% by weight of said cationic or cationizable polyurethane.

38. The cosmetic composition according to claim 36, wherein it is a nail polish and comprises from 0.5 to 40% by weight of said cationic or cationizable polyurethane.

39. The cosmetic composition according to claim 36, wherein it is a make-up composition for skin, lips and skin appendages and comprises from 0.5 to 20% by weight of said cationic or cationizable polyurethane.

40. A process for forming a protecting film on nails, said process comprising applying to nails a cationic or cationizable polyurethane with elastic character formed by the reaction of:
(a1) at least one cationic or cationizable unit resulting from at least one tertiary or quaternary amine having at least two reactive functions with labile H,
(a2) at least one mixture of at least two different non-ionic units having at least two reactive functions with labile H,
(b) at least one compound comprising at least two isocyanate functions, wherein the at least one mixture of non-ionic units (a2) comprises a mixture of
at least one first non-ionic polymer chosen from olefin homopolymers and copolymers, and
at least one second non-ionic polymer different from an olefin copolymer and chosen from non-ionic homopolymers and copolymers, and
further wherein said at least one first non-ionic polymer and said at least one second non-ionic polymer bear at their ends reactive functions with labile hydrogen and having a glass transition temperature (Tg), measured by differential scanning calorimetry, less than 10° C.

41. A process for permanent styling of hair, said process comprising applying to said hair a cationic or cationizable polyurethane with elastic character formed by the reaction of:
(a1) at least one cationic or cationizable unit resulting from at least one tertiary or quaternary amine having at least two reactive functions with labile H,
(a2) at least one mixture of at least two different non-ionic units having at least two reactive functions with labile H,
(b) at least one compound comprising at least two isocyanate functions, wherein the at least one mixture of non-ionic units (a2) comprises a mixture of
at least one first non-ionic polymer chosen from olefin homopolymers and copolymers, and
at least one second non-ionic polymer different from an olefin copolymer and chosen from non-ionic homopolymers and copolymers, and
further wherein said at least one first non-ionic polymer and said at least one second non-ionic polymer bear at their ends reactive functions with labile hydrogen and having a glass transition temperature (Tg), measured by differential scanning calorimetry, less than 10° C.

42. A process for making a cosmetic composition in a form chosen from styling lacquers, mousses and gels, said process comprising including in said cosmetic composition a cationic or cationizable polyurethane with elastic character formed by the reaction of:
(a1) at least one cationic or cationizable unit resulting from at least one tertiary or quaternary amine having at least two reactive functions with labile H,
(a2) at least one mixture of at least two different non-ionic units having at least two reactive functions with labile H,
(b) at least one compound comprising at least two isocyanate functions, wherein the at least one mixture of non-ionic units (a2) comprises a mixture of
at least one first non-ionic polymer chosen from olefin homopolymers and copolymers, and
at least one second non-ionic polymer different from an olefin copolymer and chosen from non-ionic homopolymers and copolymers, and
further wherein said at least one first non-ionic polymer and said at least one second non-ionic polymer bear at their ends reactive functions with labile hydrogen and having a glass transition temperature (Tg), measured by differential scanning calorimetry, less than 10° C.

43. A process for cosmetically treating hair, said process comprising
applying to hair a cationic or cationizable polyurethane with elastic character formed by the reaction of:
(a1) at least one cationic or cationizable unit resulting from at least one tertiary or quaternary amine having at least two reactive functions with labile H,
(a2) at least one mixture of at least two different non-ionic units having at least two reactive functions with labile H,
(b) at least one compound comprising at least two isocyanate functions, wherein the at least one mixture of non-ionic units (a2) comprises a mixture of
at least one first non-ionic polymer chosen from olefin homopolymers and copolymers, and
at least one second non-ionic polymer different from an olefin copolymer and chosen from non-ionic homopolymers and copolymers, and
further wherein said at least one first non-ionic polymer and said at least one second non-ionic polymer bear at their ends reactive functions with labile hydrogen and having a glass transition temperature (Tg), measured by differential scanning calorimetry, less than 10° C.
